(19) United States
(12) Patent Application Publication
(10) Pub. No.: US 2011/0308532 A1
(43) Pub. Date: Dec. 22, 2011

(54) Apparatus for Clamping on the Lower or Upper Teeth, and Activator Comprising Such an Apparatus

(76) Inventor: Frans Jozef Nelissen, Nijlen (BE)

(21) Appl. No.: 13/144,255

(22) PCT Filed: Jan. 8, 2010
(86) PCT No.: PCT/BE10/00001

§ 371 Date: Sep. 8, 2011

(30) Foreign Application Priority Data
Jan. 13, 2009 (BE) 2009/0016

(51) Int. Cl. A61F 5/56

(52) U.S. Cl. 128/848

(57) ABSTRACT

Apparatus which can be placed clampingly on a lower jaw or upper jaw, in particular on the molars and/or pre-molars thereof, essentially consisting of:—at least a left and a right buccal shaped body on the buccal side;—one or more connecting elements between the left and right buccal shaped body, wherein the left and right buccal shaped bodies extend at least along respectively the left and right molars and/or premolars and/or canines; and wherein the one or more connecting elements are adapted to push the left and right shaped bodies into the undercuts of the molars and/or premolars and/or canines.

The present invention relates to an apparatus which can be placed clampingly on the teeth of a lower or upper jaw. Such apparatus can be utilized for different applications, for instance for holding the lower jaw forward relative to the upper jaw, particularly for the purpose of treating nighttime breathing problems. The invention therefore also relates to a device for use in such an application, comprising an apparatus according to the invention.

The existing braces which can be arranged clampingly on the teeth typically consist of one plastic shaped body in which the shape of the teeth is arranged such that this shaped body can be placed on the teeth. A distinction can be made here between the full braces which extend over substantially all teeth and the braces in which a part is omitted at the front on the outside and at the rear on the inside in order to improve the wearing comfort. The full braces have the drawback that it is difficult to find a suitable balance in respect of the undercut (sufficient/insufficient undercut). Although the omission of parts of material of the brace on the outside at the front and on the inside at the rear makes the brace more pleasant to wear, it has the drawback that the brace will break more easily.

US 2005/0011525 discloses an adjuster device including a first bite body 16 and a second bite body 20. The first bite body 16 includes molar anchors 100, 102 for maintaining the first bite body in place on the teeth. Note that bridge 96 is not suitable for this task. Similarly, second bite body 20 includes molar anchors 92 for maintaining the second bite body in place on the teeth, second frame 88 not being suitable for this task.

The present invention has for its object to provide an apparatus of the type stated in the preamble, which is more user-friendly and in particular more pleasant to wear, and which is at the same time robust, simple to manufacture and provides a controllable flexibility, while the apparatus can still be anchored firmly on a dental arch.

The apparatus according to the preamble is distinguished for this purpose in that this apparatus comprises:
at least a left and a right shaped body on the buccal or lingual side;
one or more connecting elements between the left and right shaped body,

wherein the left and right shaped bodies extend at least along undercuts of respectively the left and right molars and/or premolars and/or canines;

and wherein the one or more connecting elements are adapted to push the left and right shaped bodies into the undercuts of the molars and/or premolars and/or canines, whereby the apparatus remains in place at typical intra-maxillary tensile forces.

By on the one hand providing at least a left and a right shaped body, which are typically manufactured substantially from a rigid plastic (typically a baseplate synthetic resin) or from a suitable metal, and then connecting these shaped bodies using connecting means which can bring about a clamping action, the volume of the material of the apparatus is limited. Robust connecting elements can be provided which can be readily adjusted to the mouth of the patient. Instead of bridging pieces integral with the shaped bodies as in the prior art, use is thus made here of distinct connecting elements which can bring about a clamping action and are readily adjustable to the teeth. In this way an apparatus is thus obtained with more ‘controlled’ flexibility compared to the prior art oral apparatus or braces or activators, wherein the apparatus can be anchored on the dental arches in very rigid/consolidated manner and provides sufficient resistance to typical intra-maxillary tensile forces, i.e. tensile forces between lower and upper jaw which are for instance exerted when the apparatus is used in an activator—see below. Apparatus are known in the prior art with flexible shaped bodies from a more flexible and bendable plastic, but these can absorb only intermaxillary tensile forces and provide insufficient resistance to intramaxillary tensile forces.

Note that the term shaped body must be broadly interpreted, and shaped body must particularly be understood to mean a random body which can consist of a plurality of mutually connected parts and which is formed to be placed against a side of the teeth, and which is thus adapted to the shape of the mouth and of the teeth. The shaped bodies preferably extend from the gums to a position at least roughly halfway along the crown of a tooth. The combination of shaped bodies and connecting elements must more particularly be able to ensure the necessary combination of retention, flexibility and stability, and for determined embodiments a height from the gums to a position for instance halfway along the tooth can suffice.

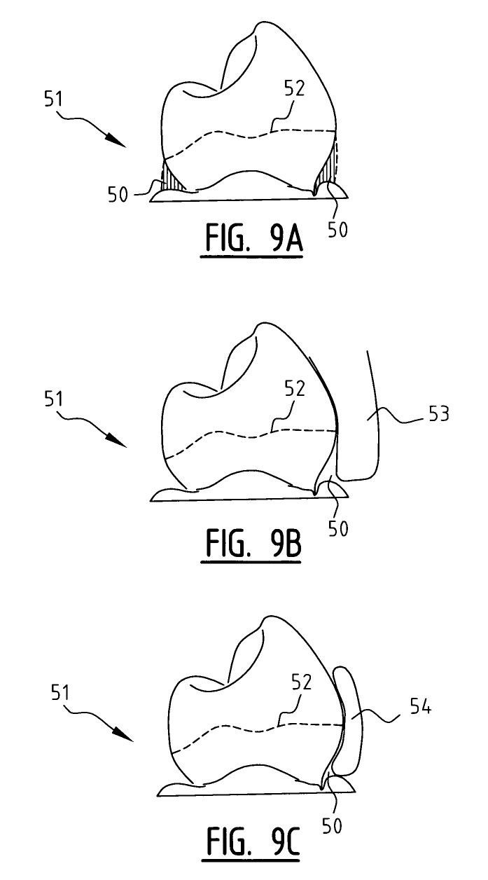

The term undercut must be understood to mean the undercut area of the crown of a tooth. This area is illustrated in FIG. 9A, see the hatched area 50 below equator 52 of tooth 51. When the prior art apparatus are made, a block-out material such as wax is placed in the undercuts to prevent the brace extending too far into the undercuts—in the case of a full brace it would after all not be easy to remove it from the teeth, or the brace would break when placed on the teeth if the material of the brace extends too deeply into the undercuts. An example is shown in FIG. 9B, where the brace does not extend into undercut 50. With an apparatus according to the invention considerably more material will typically be placed in undercuts 50 than previously, wherein this material in combination with the connecting elements provides for a higher retention, i.e. support against tensile forces, of the apparatus in the mouth. This is illustrated in FIG. 9C, which shows that a shaped body 54 extends far into undercut 50.

According to a possible embodiment, an apparatus according to the invention comprises:
a lingual shaped body on the lingual side;
at least one buccal shaped body on the buccal side;
a left and a right connecting element between the lingual shaped body on the one hand and the at least one buccal shaped body on the other, which left and right connecting elements are located on sides of the mouth laterally opposite each other and are adapted to clamp the lingual shaped body and the at least one buccal shaped body against the teeth.

By providing a lingual shaped body on the one hand and at least one buccal shaped body on the other, typically manufactured substantially from plastic or from metal, and by then connecting these shaped bodies using left and right connecting means which can bring about a clamping action, the volume of the material of the apparatus is limited. Instead of bridge pieces integral with the shaped bodies as in the prior art, use is thus made here of distinct left and right connecting elements which can bring about a clamping action and are readily adjustable to the teeth.

According to another possible embodiment, no components are necessary on the lingual side and the apparatus consists essentially of:
at least a left and a right buccal shaped body on the buccal side;
one or more connecting elements between the left and right buccal shaped body, these connecting elements running along the buccal side of the front teeth.

The so-called ‘retention’ principle of the apparatus without a lingual component is based on, among others, the following facts: the reverse conical shape of the crowns of the teeth which are to some extent divergent provides ‘undercut’ (retention), wherein the hardness of the part of the shaped bodies placed in this ‘undercut’ provides ‘stable grip’. The flexibility needed to push these rigid parts in each case into these undercuts is realized by the connecting element mutually connecting the rigid shaped bodies.

According to yet another possible embodiment, no components are necessary on the buccal side and the apparatus consists essentially of:
at least a left and a right lingual shaped body on the lingual side;
one or more connecting elements between the left and the right lingual shaped bodies, these connecting elements running along the lingual side, preferably close to the front teeth.

According to a preferred embodiment, the entirety of connecting elements between the left and right shaped bodies is more flexible and less brittle than the shaped bodies themselves. One or more connecting elements can for instance be provided in wire or strip form, which imparts flexibility. According to an advantageous embodiment, each connecting wire or strip is formed from at least one bendable metal strip or metal wire.

According to an advantageous embodiment, the at least one buccal shaped body and/or the lingual shaped body/bodies are manufactured substantially from a rigid plastic. According to another option, these shaped bodies are manufactured substantially from metal or from a combination of metal and plastic. Each shaped body is preferably manufactured from a baseplate synthetic resin (splint synthetic resin) on the basis of for instance UDMA, PMMA, PC or a combination thereof, and for instance Eclipse synthetic resin from the company Dentsply. The baseplate synthetic resin used is preferably light-curable. The shaped bodies preferably have a hardness comparable to the hardness of the light-cured Eclipse synthetic resin from Dentsply (USA).

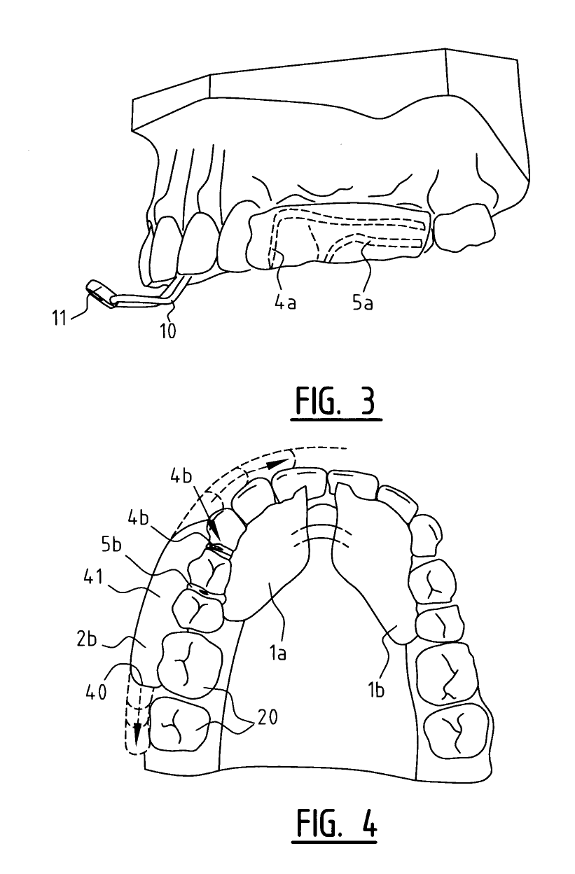

According to the preferred embodiment of the invention, the left and right shaped bodies are manufactured integrally, see also the figure description below with reference to FIG. 4.

For the left and right shaped bodies it is particularly important that a good grip is obtained by these shaped bodies on the teeth. These shaped bodies therefore preferably extend over teeth with a sufficient roundness, such as the molars and the premolars.

According to a further developed embodiment, the left and the right shaped bodies extend respectively at least along a left premolar and along a right premolar, and/or respectively over the distal side of a left canine and a right canine, and/or respectively from the distal side of a left canine to a left molar and from the distal side of a right canine to a right molar. The left and right shaped bodies still more preferably extend over at least a part of the molars and premolars.

The skilled person will appreciate that more than two left or right shaped bodies can be arranged distributed against the outside of the teeth, wherein the different buccal shaped bodies can optionally be connected to each other, for instance using metal wires.

The connecting elements are for instance formed in each case by at least two wires, preferably metal wires. In the embodiment with buccal and lingual components the wires can extend occlusally or interproximally between the lingual shaped body and the at least one buccal shaped body. According to a possible embodiment, a first wire of each connecting means extends on the distal side of premolar, preferably the foremost premolar, and a second wire of each connecting means extends on the mesial side of this premolar.

As stated in the preamble, an apparatus as described above can be used for different applications, and particularly for the purpose of treating breathing problems. Nighttime breathing problems, which can for instance result in snoring, the sleep apnoea syndrome or other sleep disorders, are a generally known problem. When a person sleeps, the rear part of the tongue may tend to slide backwards and so wholly or partially close off the pharyngeal airway. It is known to solve such breathing problems with a device which can be placed in the mouth and with which the lower jaw is placed further forward in relation to the upper jaw.

The device according to the invention, in particular for treating breathing problems, is distinguished in that it comprises a lower apparatus as described above fitting onto the teeth of the lower jaw, an upper apparatus as described above fitting onto the teeth of the upper jaw, and connecting means between the upper and lower apparatus. The connecting means are preferably adjustable.

According to a preferred embodiment of such a device, the connecting means are adapted to adjust the upper apparatus forward/rearward and/or upward/downward relative to the lower apparatus. Such connecting means are known and are for instance discussed in patent specification WO 2004/000112 and in the Belgian patent application BE 2008/0374 in the name of applicant. Other connecting means are further also known, particularly from patent specifications in the name of Hallstrom and Thornton, which can also be used in a device according to the invention.

According to a preferred embodiment of the device according to the invention, the shaped body of the upper or the lower apparatus is provided at the front with a guide element for a coupling element which is slidable laterally along this guide element and which can be coupled to the shaped body of respectively the lower or upper apparatus. The coupling element is more preferably provided with a screw thread and the shaped body of the lower or upper apparatus is provided with a threaded bore which can co-act with the screw thread for the purpose of adjusting the lower apparatus upward/downward relative to the upper apparatus.

The invention will be further elucidated with reference to the appended drawings and the associated figure description of a number of non-limitative exemplary embodiments of an apparatus and device according to the invention. In the drawing:

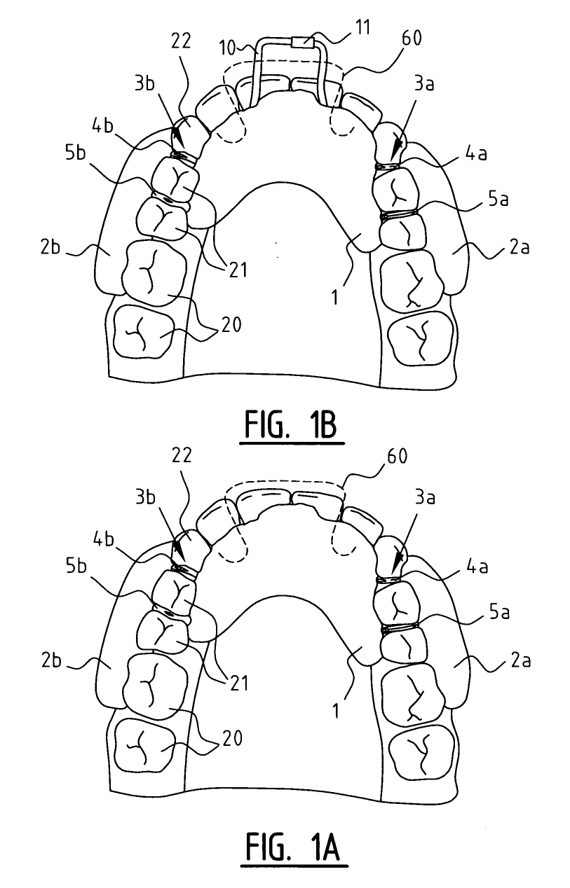

FIG. 1A is a bottom view of a first possible embodiment of an apparatus according to the invention for placing on the upper teeth;

FIG. 1B is a bottom view similar to that of FIG. 1A, but in which the apparatus is additionally adapted for use in an activator;

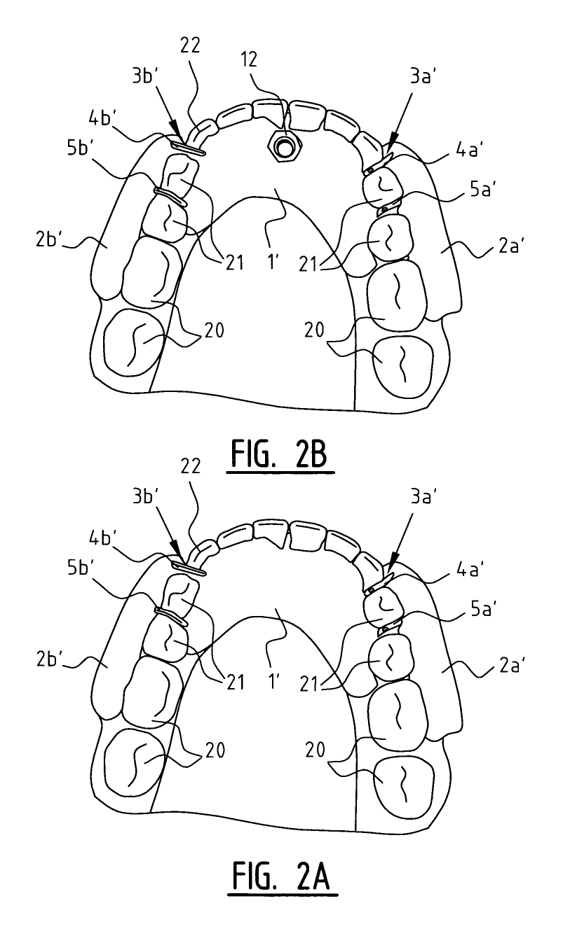

FIG. 2A is a bottom view of a second possible embodiment of an apparatus according to the invention for placing on the lower teeth;

FIG. 2B is a bottom view similar to that of FIG. 2A, but in which the apparatus is additionally adapted for use in an activator;

FIG. 3 is a perspective side view of the embodiment of FIG. 1B;

FIG. 4 is a schematic bottom view of the upper or lower teeth on which a third embodiment of an apparatus according to the invention is arranged;

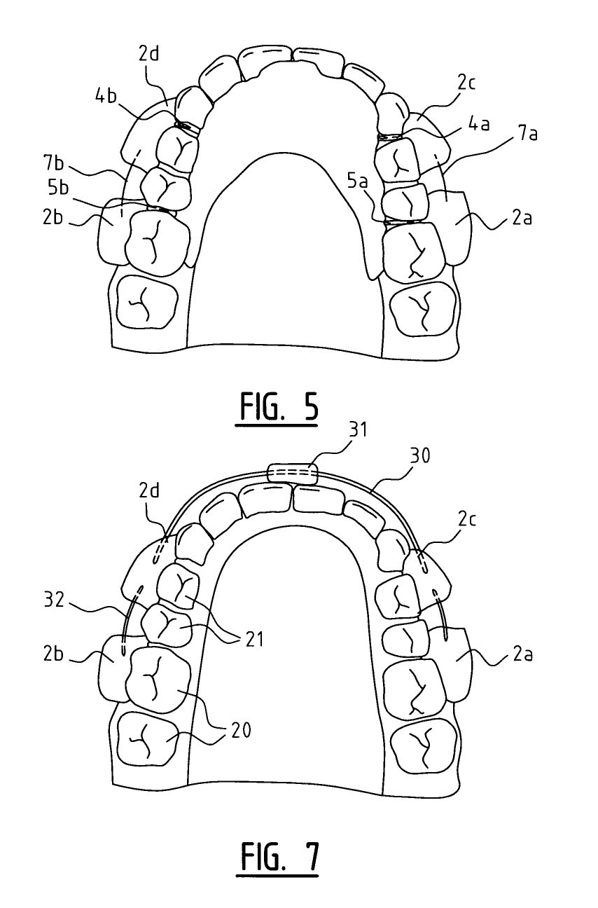

FIG. 5 is a schematic bottom view of the upper or lower teeth on which a fourth embodiment of an apparatus according to the invention is arranged;

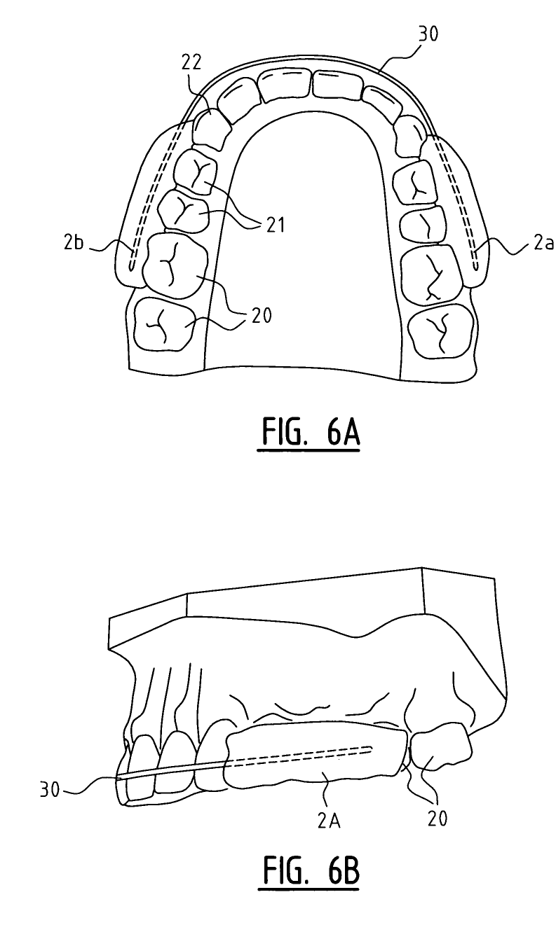

FIG. 6 is a bottom view of a fifth possible embodiment of an apparatus according to the invention for placing on the upper or lower teeth;

FIG. 6B is a perspective side view of the embodiment of FIG. 6A;

FIG. 7 is a bottom view of a sixth possible embodiment of an apparatus according to the invention for placing on the upper or lower teeth;

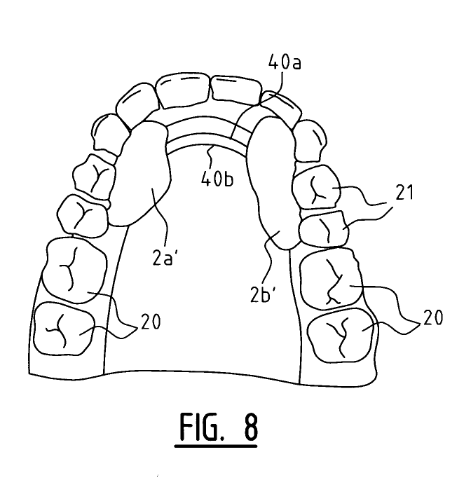

FIG. 8 is a schematic bottom view of the upper or lower teeth on which a seventh embodiment of an apparatus according to the invention is arranged.

A first and second embodiment of a device according to the invention is shown in FIGS. 1-3. FIGS. 1A/1B show an apparatus which can be placed clampingly on the teeth of an upper jaw. The apparatus comprises:
on the lingual side a lingual shaped body 1 manufactured from plastic, typically a monomer-free plastic, and for instance from Eclipse material from the Dentsply company; note that this shaped body could also be manufactured from metal, if the metal were made sufficiently thick to obtain the desired rigidity of the shaped body;
on the buccal side a right buccal shaped body 2a and a left buccal shaped body 2b, also manufactured from plastic, typically a monomer-free plastic; note that this shaped body could also be manufactured from metal;
a left and a right connecting element 3a, 3b between lingual shaped body 1 on the one hand and the left and right buccal shaped bodies 2a, 2b on the other, which left and right buccal shaped bodies lie laterally opposite each other on a left and a right-hand side of the mouth. These shaped bodies 1, 2a, 2b are adapted to the shape of the teeth such that sufficient grip is realized and the apparatus can be clamped firmly on the upper jaw.

The left and right buccal shaped bodies 2a, 2b extends along:
respectively the distal side of a left canine 22b and a right canine 22a;
respectively a left front and rear premolar 21b and a front and rear right premolar 21a;
respectively a left front molar 22b and a right front molar 22a.

The skilled person will however appreciate that it is possible to embody the at least one buccal shaped body in other manner, as will be further elucidated with reference to FIGS. 4 and 5.

Each connecting means 3a, 3b consists here of a first wire 4a, 4b and a second wire 5a, 5b, which first and second wires run interproximally on either side of the foremost premolar. The skilled person will however appreciate that the wires can run occlusally or interproximally over the teeth between lingual shaped body 1 and the at least one buccal shaped body 2a, 2b. Wires 4a, b and 5a, b are preferably metal wires, although any type of wire which is bendable and not brittle and which can bring about a clamping action is in principle also suitable. Nylon, plastic or glass fibre wires are also suitable.

FIGS. 2A/B show an apparatus which can be placed clampingly on the teeth of a lower jaw. The apparatus is similar to that of FIGS. 1A/B, with the difference that the shaped bodies are adjusted to the teeth of the lower jaw. Similar components are designated with the same reference numerals.

When the upper and lower apparatus shown in FIGS. 1A and 2A are used to move forward and hold the lower jaw relative to the upper jaw, connecting means are typically then arranged between the upper and the lower apparatus. According to a possible embodiment as shown in FIGS. 1B and 2B, lingual shaped body 1 of the upper apparatus is provided at the front with a guide element 10 for a coupling element 11 which is slidable laterally along this guide element. This coupling element 11 can be provided with a screw which can co-act with a threaded bore 12 in lingual shaped body 1′ of the lower apparatus such that the distance between the upper and the lower apparatus can be adjusted.

According to a possible embodiment, an anti-bruxism rail 60 (shown in broken lines in FIGS. 1A and 1B) can optionally be provided. This consists here of a wire extending from a first position on lingual shaped body 1 over the front teeth to a second position on the lingual shaped body. The anti-bruxism rail is positioned such that grinding of the teeth is prevented.

The apparatus according to the invention can also be referred to with the terms retainer or clip, this indicating the clamping action of the apparatus on the teeth.

FIG. 4 shows schematically a third embodiment of an apparatus according to the invention. The lingual shaped body is formed here from two parts 1a, 1b, which can for instance be manufactured from plastic or metal. These two parts are mutually connected by two connecting wires 6. FIG. 5 illustrates that buccal shaped body 2b can have variable dimensions and that outer ends 40, 41 of this shaped body can extend to respectively the molars and the front teeth. According to a possible embodiment, the left and the right buccal shaped bodies 2a, 2b will form one part which extends along the front teeth.

FIG. 5 illustrates a fourth embodiment in which four buccal shaped bodies 2a-2d are provided. A first pair of buccal shaped bodies 2b, 2d are located on the left side of the mouth and are mutually connected by a connecting wire 7b. In similar manner a second pair of shaped bodies 2a, 2c are located on the right side of the mouth and mutually connected by a connecting wire 7a. The skilled person will understand that even more shaped bodies can be provided. What is most important is that the buccal shaped bodies have sufficient grip on the teeth.

In the embodiment of FIG. 5 the connecting means comprise two sets of connecting wires, wherein each set comprises: an interproximal connecting wire 4a, 4b between a foremost premolar and a canine, and an occlusal connecting wire 5a, 5b over a rear premolar. The skilled person will once again understand that other numbers and combinations of connecting wires are possible.

FIGS. 6A and B illustrate a fifth embodiment of an apparatus according to the invention. The apparatus consists here of:—on the buccal side a left and a right buccal shaped body 2a, 2b, manufactured for instance from a hard plastic;—a connecting element between the left and right buccal shaped bodies in the form of connecting wire 30, which extends along the buccal side of the front teeth. This flexible metal connecting wire 30 will ensure that the left and right shaped bodies 2a, 2b are pushed into the undercuts of foremost molars 20, premolars 21 and some of the canines 22.

FIG. 7 illustrates a sixth embodiment which has some similarity to the fifth embodiment, with the difference that the two left and two right buccal shaped bodies 2a, 2c and 2b, 2d are provided, which are connected to each other, in each case by a connecting wire 32. A rear buccal shaped body 2a, 2b will for instance engage against molars 20 and a front buccal shaped body against premolars 21. An additional shaped body 31 can further be provided which supports against the front teeth, wherein connecting wire 30 runs through this shaped body 31.

FIG. 8 illustrates a seventh embodiment, in which use is made solely of lingual components. The apparatus is provided here with a left and a right lingual component 2a′, 2b′, which are connected to each other by means of two connecting wires 40a, 40b extending on the lingual side close to the front teeth. In the shown embodiment lingual shaped bodies 2a′, 2b′ engage in the undercuts of foremost molars 20, premolars 21 and canines 22. The skilled person will however appreciate that variations hereof are possible, wherein shaped body 2a′ or 2b′ can for instance consist of multiple pieces mutually connected via connecting wires.

The present invention is not limited to the above described exemplary embodiments, and the skilled person will appreciate that many modifications are possible, such as providing a plurality of buccal shaped bodies or a multi-part lingual shaped body. The scope of protection is therefore defined solely by the following claims.

1.-21. (canceled)

22. An apparatus which can be placed clampingly on a lower or upper jaw, in particular on the molars and/or premolars thereof, essentially consisting of:
at least a left and a right shaped body (2a, 2b; 2a′, 2b′) on buccal or on lingual side;
one or more connecting elements between the left and the right shaped body (4a-b, 5a-b, 1; 30; 40a-b),
wherein the left and right shaped bodies extend at least along respectively the left and right molars and/or premolars and/or canines; and
wherein the one or more connecting elements are adapted to push the left and right shaped bodies into the undercuts of the molars and/or premolars and/or canines, and to hold the apparatus in place via a clamping action at typical intra-maxillary tensile forces.

23. An apparatus which can be placed clampingly on a lower or upper jaw, in particular on the molars and/or premolars thereof, comprising:
a lingual shaped body on the lingual side;
at least one buccal shaped body on the buccal side;
a left and a right connecting element between the lingual shaped body on the one hand and the at least one buccal shaped body on the other, which left and right connecting elements are located on sides of the mouth laterally opposite each other and are adapted to clamp the lingual shaped body and the at least one buccal shaped body against the teeth.

24. The apparatus of claim 22, wherein the entirety of the one or more connecting elements takes a more flexible form than the at least one left and right shaped body, and that each shaped body is a rigid body.

25. The apparatus of claim 22, wherein the at least one left and right shaped body are buccal shaped bodies on the buccal side, and that the one or more connecting elements comprise:
a lingual shaped body (1) on the lingual side;
at least one left and one right connecting wire or strip (4a-b) between the lingual shaped body on the one hand and the at least one buccal shaped body on the other, which left and right connecting wires or strips are located on sides of the mouth laterally opposite each other and are adapted to clamp the lingual shaped body and the at least one buccal shaped body against the teeth.

26. The apparatus of claim 22, wherein the at least one left and right shaped body are buccal shaped bodies on the buccal side, and that the one or more connecting elements comprise one or more connecting wires or strips (30) extending on the buccal side of the front teeth, and preferably consist of one connecting wire or strip extending along the buccal side of the front teeth.

27. The apparatus of claim 22, wherein the at least one left and right shaped body are lingual shaped bodies on the lingual side, and that the one or more connecting elements comprise one or more connecting wires or strips (4a-b) extending on the lingual side, preferably close to the front teeth.

28. The apparatus of claim 25, wherein the at least one left and right connecting wire or strip (4a-b) is formed in order to press respectively the left and right buccal shaped bodies against the buccal side of the teeth and to press the lingual shaped body against the lingual side of the teeth.

29. The apparatus of claim 25, wherein the at least one left and right connecting wire or strip extend occlusally or interproximally over the teeth between the lingual shaped body on the one hand and respectively the left or right buccal shaped bodies on the other.

30. The apparatus of claim 25, wherein the at least one left and right connecting wire or strip (4a, 4b; 5a, 5b) comprises in each case at least two wires or strips.

31. The apparatus of claim 30, wherein a first wire or strip of each at least two wires or strips extends on the distal side of premolar, preferably the foremost premolar, and a second wire of each connecting means extends on the mesial side of this premolar.

32. The apparatus of claim 22, wherein each connecting wire or strip is formed from at least one bendable metal strip, preferably a metal wire.

33. The apparatus of claim 22, wherein each shaped body is manufactured substantially from a rigid plastic or from metal, and preferably from a baseplate synthetic resin, more preferably on the basis of UDMA, PMMA, PC or a combination thereof, and for instance Eclipse synthetic resin from the company Dentsply, or a plastic material with a comparable hardness in cured form.

34. The apparatus of claim 22, wherein the at least one left and the at least one right shaped body extend respectively at least along a left premolar and along a right premolar; and/or over the distal side of a left canine and a right canine.

35. The apparatus of claim 22, wherein the at least one left and the at least one right shaped body at least extend respectively from the distal side of a left canine to a left molar and from the distal side of a right canine to a right molar.

36. The apparatus of claim 22, wherein the left and right shaped bodies extend at least along respectively the left and right molars and/or premolars and/or canines; and
wherein the one or more connecting elements are adapted to push the left and right shaped bodies into the undercuts of the molars and/or premolars and/or canines, and to hold the apparatus in place via a clamping action at typical intra-maxillary tensile forces; and
wherein the at least one left and right shaped body are buccal shaped bodies on the buccal side, and the one or more connecting elements comprise:
a lingual shaped body (1) on the lingual side,;
at least one left and one right connecting wire or strip (4a-b) between the lingual shaped body on the one hand and the at least one buccal shaped body on the other, which left and right connecting wires or strips are located on sides of the mouth laterally opposite each other and are adapted to clamp the lingual shaped body and the at least one buccal shaped body against the teeth.

37. The apparatus of claim 36, wherein the at least one left and right connecting wire or strip extend occlusally or interproximally over the teeth between the lingual shaped body on the one hand and respectively the left or right buccal shaped bodies on the other.

38. A device comprising a lower apparatus as claimed in claim 22 fitting onto the teeth of the lower jaw, an upper apparatus as claimed in claim 22 fitting onto the teeth of the upper jaw, and connecting means between the upper and lower apparatus.

39. The device of claim 38, wherein the connecting means are adjustable.

40. The device of claim 39, wherein the connecting means are adapted to adjust the upper apparatus forward/rearward and/or upward/downward relative to the lower apparatus.

41. The device of claim 38, wherein a shaped body of the upper or the lower apparatus is provided with a guide element for a coupling element which is slidable laterally along this guide element and which can be coupled to a shaped body of respectively the lower or upper apparatus.
